(19) United States
(12) Patent Application Publication
(10) Pub. No.: US 2004/0204603 A1
(43) Pub. Date: Oct. 14, 2004

(54) Process for the hemihydrogenation of dinitriles to form aminonitriles

(76) Inventors: Philippe Leconte, Meyzieu (FR); Joseph Lopez, Villeurbanne (FR)

Correspondence Address: BURNS DOANE SWECKER & MATHIS L L P, POST OFFICE BOX 1404, ALEXANDRIA, VA, 22313-1404, US

(21) Appl. No.: 10/481,028

(22) PCT Filed: Jun. 13, 2002
(86) PCT No.: PCT/FR02/02023

(30) Foreign Application Priority Data
Jun. 22, 2001 (FR) 01/08245

(51) Int. Cl. C07C 253/30

(52) U.S. Cl. 558/459

(57) ABSTRACT

The invention relates to the hemihydrogenation of dinitriles to form corresponding aminonitriles. The invention more particularly relates to a method for the hemihydrogenation of dinitriles in the presence of water and selectifying agents enabling aminonitrile selectivity to be improved.

The present invention relates to the hemihydrogenation of dinitriles to corresponding aminonitriles.

The hydrogenation of dinitriles is generally carried out in order to prepare the corresponding diamines; thus, particularly, the hydrogenation of adiponitrile results in hexamethylenediamine, itself one of the two base compounds for the preparation of polyamide-6,6.

However, it can sometimes prove necessary to prepare not the diamine but the intermediate aminonitrile. This is the case, for example but not limitingly, in the hemihydrogenation of adiponitrile to aminocapronitrile, which is capable of subsequently being converted to caprolactam, the base compound for polyamide-6, or directly to polyamide-6.

Thus, U.S. Pat. No. 4,389,348 discloses a process for the hydrogenation of dinitrile to ω-aminonitrile by hydrogen in ammonia and aprotic solvent medium in the presence of rhodium deposited on a basic support.

U.S. Pat. No. 5,151,543 discloses a process for the partial hydrogenation of dinitriles to aminonitriles in a solvent in molar excess of at least 2/1 with respect to the dinitrile, comprising liquid ammonia or an alkanol comprising an inorganic base which is soluble in the said alkanol, in the presence of a catalyst of Raney cobalt or nickel type.

U.S. Pat. No. 5,981,790 relates to a process for the partial hydrogenation of dinitriles to aminonitriles in the presence of a catalyst based on Raney nickel or Raney cobalt in the presence of at least 0.5% by weight of water in the reaction medium comprising the products to be hydrogenated and the hydrogenated compounds. The catalyst is used in conjunction with a base.

These various processes make it possible to jointly produce an aminonitrile and a diamine in relatively high ratios and with relatively high production of by-products which are difficult to separate. Studies are continually being carried out to modify this ratio in order in particular to increase the production of aminonitrile at the expense of that of diamine and also to reduce the formation of by-products.

Thus, Patent Application WO 00/64862 discloses a process for the partial hydrogenation of a dinitrile for the production of aminonitriles in the presence of a hydrogenation catalyst, of an alkanol or a liquid ammonia solvent and of a compound which makes it possible to improve the selectivity of the reaction for aminonitriles. However, the production of undesirable by-products remains high.

One of the objects of the present invention is to provide a novel process for the selective hydrogenation of a single nitrile functional group of a dinitrile (referred to in the present text as hemihydrogenation), so as to prepare predominantly the corresponding aminonitrile and only to a minor extent the diamine, with minimal formation of by-products.

More specifically, the invention relates to a process for the hemihydrogenation of aliphatic dinitriles to corresponding aminonitriles using hydrogen in the presence of a hydrogenation catalyst, for example based on nickel, on cobalt, on Raney nickel or on Raney cobalt, optionally comprising a doping element chosen from the elements from Groups 3 to 12 of the Periodic Table of the Elements according to the IUPAC nomenclature published in the Handbook of Chemistry and Physics, 80th edition, 1999-2000, and of a strong inorganic base deriving from an alkali metal or alkaline earth metal or from an ammonium. The starting hydrogenation medium comprises water at a content of at least 0.5% by weight with respect to all of the liquid compounds of the said medium, diamine and/or aminonitrile capable, of being formed from the dinitrile to be hydrogenated, and unconverted dinitrile, the content by weight of the combination of these three compounds in the medium being between 80% and 99.5%.

According to the invention, the hemihydrogenation reaction is carried out in the presence of at least one additive which increases the selectivity for aminonitrile with respect to that obtained with the additive-free system described above, while maintaining the overall selectivity for aminonitrile and diamine at a level at least substantially equivalent to that obtained without the additive.

The term “selectivity for a product” is understood to mean the yield of this product obtained, calculated with respect to the amount of dinitrile converted at the end of the reaction.

This additive is a compound chosen from the group consisting of:

a compound comprising at least one cyanide radical not bonded to a carbon atom,

an organic isonitrile compound,

a tetraalkylammonium or tetraalkyl-phosphonium hydroxide or fluoride compound,

a complex compound from coordination between at least one metal atom and at least carbonyl radicals,

an alkali metal or alkaline earth metal fluoride compound.

Mention may be made, as compounds comprising at least one cyanide radical not bonded to a carbon atom, of inorganic cyanides, organic/inorganic cyanides, cyanide complexes or salts, such as hydrogen cyanide, lithium, sodium, potassium or copper cyanide, the cyanide complexes K₃[Fe(CN)₆], K₄[Fe(CN)₄], K₃[Co(CN)₆], K₂[Pt(CN)₆] or K₄[Ru(CN)₆], or ammonium or alkali metal thiocyanides. Mention may be made, as organic/inorganic cyanide, of tetraalkylammonium cyanides, such as tetrabutylammonium cyanide, tetramethylammonium thiocyanide or tetrapropylammonium thiocyanide.

Mention may be made, as organic isonitrile compounds suitable for the invention, of tert-octylisonitrile, tert-butylisonitrile, n-butylisonitrile, isopropylisonitrile, benzylisonitrile, ethylisonitrile, methylisonitrile and amylisonitrile.

Mention may be made, as coordination complex compounds, of the complexes comprising, as complexing compounds, organic compounds comprising carbonyl, phosphine, arsine or mercapto functional groups connected to the metal. Mention may be made, as suitable metals, of in particular the metals from Groups 7, 8, 9 and 10 of the Periodic Table of the Elements mentioned above, such as, for example, iron, ruthenium, cobalt, osmium, rhenium, iridium or rhodium.

Mention may be made, as organic tetraalkylonium hydroxide or fluoride compounds, of tetraalkylammoniums or tetraalkylphosphoniums comprising hydroxyl groups or fluorine atoms connected to the ammonium or phosphonium groups. The alkyl radicals are preferably hydrocarbonaceous groups comprising from 1 to 8 carbon atoms. These radicals can be linear or branched. By way of examples, tetramethylammonium, tetraethylammonium, tetrapropylammonium, tetrabutylammonium and tetrabutylphosphonium are suitable for the invention.

According to a preferred characteristic of the invention, the ratio by weight of the selectivating agent with respect to the weight of catalytic element, expressed as weight of metal, such as nickel, is between 0.001:1 and 2:1, advantageously between 0.005:1 and 1:1. This ratio varies according to the nature of the selectivating agent.

The process of the invention makes it possible to obtain, for degrees of conversion of the dinitrile of greater than 70%, a selectivity for aminonitrile of greater than 65% and an overall selectivity for aminonitrile and diamine of greater than 90%.

The aliphatic dinitriles which can be employed in the process of the invention are more particularly the dinitriles of general formula (I):

NC—R—CN (I)

in which R represents a linear or branched alkylene or alkenylene group having from 1 to 12 carbon atoms.

Use is preferably made, in the process of the invention, of the dinitriles of formula (I) in which R represents a linear or branched alkylene radical having from 2 to 6 carbon atoms.

Mention may in particular be made, as examples of such dinitriles, of adiponitrile (AdN), methylglutaronitrile, ethylsuccinonitrile, malononitrile, succinonitrile, glutaronitrile and their mixtures, in particular the mixtures of adiponitrile and/or of methylglutaronitrile and/or of ethylsuccinonitrile capable of originating from the same process for the synthesis of adiponitrile.

In practice, the case where R=(CH₂)₄ will be the commonest, as this corresponds to the use of adiponitrile (AdN) in the present process.

The strong inorganic base is generally composed of alkali metal or alkaline earth metal or ammonium hydroxides, carbonates and alkoxides. It is preferably chosen from alkali metal hydroxides, carbonates and alkoxides.

Preferably, the strong inorganic base employed is chosen from the following compounds: LiOH, NaOH, KOH, RbOH, CsOH and their mixtures.

In practice, NaOH and KOH are generally used, although RbOH and CsOH can give very good results.

Water is usually present in the reaction medium in an amount of less than or equal to 20% by weight. Preferably, the content of water in the reaction medium is between 2% and 15% by weight with respect to the combined liquid consistuents of the said medium.

The overall concentration of targeted aminonitrile and/or of the corresponding diamine and of the unconverted dinitrile in the reaction medium is generally between 85% and 99% by weight with respect to the combined liquids included in the said reaction medium.

The amount of strong inorganic base is advantageously greater than or equal to 0.05 mol/kg of catalyst. Preferably, it is between 0.1 mol and 3 mol per kg of catalyst and more preferably still between 0.15 and 2 mol/kg of catalyst.

The catalyst used in the process can be nickel, cobalt, a Raney nickel or a Raney cobalt. The latter Raney metals comprise, in addition to the nickel or the cobalt and the residual amounts of the metal removed from the starting alloy during the preparation of the catalyst, that is to say generally aluminium, one or more other elements, often referred to as dopants, such as, for example, chromium, titanium, molybdenum, tungsten, iron, zinc, copper, rhodium, iridium, cobalt and nickel. Among these doping elements, chromium and/or iron and/or titanium are regarded as the most advantageous. These dopants usually represent, as weight by weight of nickel, from 0% to 10% and preferably from 0% to 5%. These dopants are also used with the catalysts based on nickel and/or on cobalt.

The amount of catalyst employed can vary very widely according in particular to the method of operation adopted or the reaction conditions chosen. By way of indication, use may be made of 0.5% to 50% by weight of catalyst with respect to the total weight of the reaction medium and generally of 1% to 35%.

According to a preferred embodiment of the invention, the catalyst is preconditioned before it is introduced into the hemihydrogenation medium. This preconditioning is advantageously carried out according to the process disclosed in the unpublished French Patent Application No. 00 02997. This process consists briefly in mixing the hydrogenation catalyst with a predetermined amount of strong inorganic base and a solvent in which the strong inorganic base is not very soluble. According to the invention, the medium comprising the catalyst thus conditioned is fed to the hydrogenation reactor, the hydrogenation reaction being carried out according to the usual conditions and procedures already disclosed in the literature.

The selectivating agent can be added to the reaction medium separately from the catalyst. In a preferred embodiment, the selectivating agent is added to the catalyst before the introduction of the latter into the reaction medium, for example at the stage of conditioning the latter.

The optimum for the selectivity for aminonitrile, at a constant degree of conversion of the dinitrile, depends on the nature and on the content of dopant, on the amount of water in the reaction medium, on the temperature and on the nature and content of base and/or selectivating agent.

The process of the invention is generally carried out at a reaction temperature of less than or equal to 150° C., preferably of less than or equal to 120° C. and more preferably still of less than or equal to 100° C.

In practical terms, this temperature is between ambient temperature (approximately 20° C.) and 100° C.

Prior to, simultaneously with or subsequent to the heating, the reaction chamber is brought to the appropriate hydrogen pressure, that is to say, in practice, between 1 bar (0.10 MPa) and 100 bar (10 MPa) and preferably between 5 bar (0.5 MPa) and 50 bar (5 MPa).

The duration of the reaction can vary as a function of the reaction conditions and of the catalyst.

In a batchwise method of operation, it can vary from a few minutes to several hours.

It should be noted that a person skilled in the art can vary the order of occurrence of the stages of the process according to the invention, according to the operating conditions.

The other conditions which govern the hydrogenation (continuous or batchwise) in accordance with the invention relate to conventional technical arrangements known per se.

The examples which follow illustrate the invention.

In these examples, the following abbreviations are used;

AdN=adiponitrile

ACN=aminocapronitrile

HMD=hexamethylenediamine

DC=degree of conversion

CY=selectivity with respect to the converted starting substrate (in this instance with respect to the AdN).

### COMPARATIVE EXAMPLE 1

The following are charged to a 100 ml stainless steel reactor equipped with a stirrer of self-suction type, with means for introducing the reactants and hydrogen and with a temperature regulation system:

| | |
|---|---|
| hexamethylenediamine | 24 g |
| water | 5.3 g |
| KOH | 0.33 mmol |
| Raney Ni (comprising 1.7% of Cr) | 0.65 g of Ni |

In this example, there is 0.5 mol KOH/kg Ni.

After having purged the reactor with nitrogen and then with hydrogen, the pressure is adjusted to 2 MPa of hydrogen. The reaction mixture is heated to 50° C.

24 g of adiponitrile are subsequently introduced instantaneously via a dropping funnel pressurized to 2.5 MPa by a pressure reducer placed on a hydrogen supply at 5 MPa. The time is taken as equal to 0 at this point. The progress of the reaction is monitored by the consumption of hydrogen in the supply, the pressure in the reactor being kept constant at 2.5 MPa, and by the analysis by gas chromatography (GC) of a withdrawn sample of the reaction mixture. When the optimum aminocapronitrile yield is reached, the reaction is halted by halting the stirring, cooling the reaction mixture and then depressurizing.

The following results are obtained:

| duration of the reaction | 33 min |
|---|---|
| DC of the AdN: | 79.6% |
| CY for ACN: | 70.1% |
| CY for HMD | 29.5% |
| CY for various other products: | 0.4% |

### EXAMPLE 2

Example 1 is repeated but charging the following reactants:

| | |
|---|---|
| hexamethylenediamine | 24 g |
| water | 5.3 g |
| KOH | 0.18 mmol |
| Raney Ni (comprising 1.7% of Cr) | 0.65 g of Ni |
| Selectivating agent (C₂H₅)₄N⁺F⁻.H₂O | 1.09 mmol (0.162 g) |

In this example, there are 0.3 mol KOH/kg Ni and a selectivating agent/nickel ratio equal to 0.25:1.

The results obtained are as follows:

| duration of the reaction | 63 min |
|---|---|
| DC of the AdN: | 83.8% |
| CY for ACN: | 81.6% |
| CY for HMD | 17.8% |
| CY for various other products: | 0.6% |

This test shows the improvement in the selectivity for ACN and a stability in the selectivity for by-products.

1: Process for the hemihydrogenation of dinitriles to corresponding aminonitriles using hydrogen in the presence of a catalyst based on nickel, on cobalt, on Raney nickel or on Raney cobalt, optionally comprising a doping element selected from the elements from Groups 3 to 12 of the Periodic Table of the Elements according to the IUPAC nomenclature published in the Handbook of Chemistry and Physics, 80th edition, 1999-2000, and of a strong inorganic base deriving from an alkali metal or alkaline earth metal or ammonia, the starting hydrogenation medium comprising water at a concentration by weight of at least 0.5% by weight with respect to all of the liquid compounds of the said medium, diamine and/or aminonitrile capable of being formed from the dinitrile to be hydrogenated, and unconverted dinitrile, the concentration by weight of the combination of these three compounds being from 80% to 99.5%, wherein the hemihydrogenation reaction is carried out in the presence of at least one selectivating agent selected from the group consisting of:
a compound comprising at least one cyanide radical not bonded to a carbon atom,
an organic isonitrile compound,
a tetraalkylammonium or tetraalkylphosphonium hydroxide or fluoride compound,
a complex compound from coordination between at least one metal atom and at least carbonyl radicals,
an alkali metal or alkaline earth metal fluoride compound.

2: Process according to claim 1, wherein the inorganic base is selected from alkali metal, alkaline earth metal or ammonium hydroxides, carbonates and alkoxides.

3: Process according to claim 1, wherein the strong inorganic base employed is selected from the following compounds: LiOH, NaOH, KOH, RbOH, CsOH and their mixtures.

4: Process according to claim 1, wherein the amount of inorganic base present in the reaction medium is greater than or equal to 0.05 mol per kilogram of catalyst.

5: Process according to claim 1, wherein the aliphatic dinitriles are the dinitriles of general formula (I):
NC—R—CN (I)
in which R represents a linear or branched alkylene or alkenylene group having from 1 to 12 carbon atoms.

6: Process according to claim 1, wherein water is present in the reaction medium in an amount of less than or equal to 20% with respect to the combined liquid constituents of said medium.

7: Process according to claim 1, wherein the concentration of the targeted aminonitrile and/or of the corresponding diamine and of the unconverted dinitrile in the reaction medium is between 85% and 99% by weight with respect to the combined liquids constituting said reaction medium.

8: Process according to claim 1, wherein the catalyst used is selected from a Raney nickel, a Raney cobalt, a Raney nickel and a Raney cobalt comprising one or more other doping elements, selected from the group consisting of chromium, titanium, molybdenum, tungsten, iron, zinc, copper, rhodium, iridium, cobalt or nickel.

9: Process according to claim 1, wherein the catalyst used is selected from a Raney nickel comprising at least one doping element selected from chromium and/or iron and/or titanium.

10: Process according to claim 1, wherein the catalyst used is selected from a Raney nickel comprising at least one doping element in a proportion of 0% to 10%.

11: Process according to claim 1, wherein the catalyst employed represents from 0.5% to 50% by weight with respect to the total weight of the reaction medium.

12: Process according to claim 1, which is carried out at a reaction temperature of less than or equal to 150° C.

13: Process according to claim 1, which is carried out at a hydrogen pressure of between 1 bar (0.10 MPa) and 100 bar (10 MPa).

14: Process according to claim 1, wherein the ratio by weight of the selectivating agent to the catalyst, expressed as weight of catalytic metal element, is between 0.001:1 and 2:1.

15: Process according to claim 1, wherein the selectivating agent is a compound comprising at least one cyanide radical not bonded to a carbon atom selected from the group consisting of hydrogen cyanide, lithium, sodium, potassium or copper cyanide, the cyanide complexes K₃[Fe(CN)₆], K₄[Fe(CN)₄], K₃[Co(CN)₆], K₂[Pt(CN)₆] and K₄[Ru(CN)₆], ammonium or alkali metal thiocyanides, tetrabutylammonium cyanide, tetramethylammonium thiocyanide and tetrapropylammonium thiocyanide.

16: Process according to claim 1, wherein the selectivating agent is an organic isonitrile compound selected from the group consisting of tert-octylisonitrile, tert-butylisonitrile, n-butylisonitrile, isopropylisonitrile, benzylisonitrile, ethylisonitrile, methylisonitrile and amylisonitrile.

17: Process according to claim 1, wherein the selectivating agent is a tetraalkylammonium or tetraalkylphosphonium hydroxide or fluoride compound selected from the group consisting of tetramethylammoniuin, tetraethylammonium, tetrapropylammonium, tetrabutylammonium and tetrabutylphosphonium.

18: Process according to claim 1, wherein the selectivating agent is a complex compound from coordination between at least one metal atom and at least carbonyl radical selected from the group consisting of organic compounds comprising carbonyl, phosphine, arsine and mercapto functional groups connected to a metal selected from the group consisting of iron, ruthenium, cobalt, osmium, rhenium, iridium and rhodium.
